(19) United States
(12) Patent Application Publication
(10) Pub. No.: US 2013/0137899 A1
(43) Pub. Date: May 30, 2013

(54) PROCESS FOR PRODUCING FLUORINE-CONTAINING SULFONYLIMIDE COMPOUND

(71) Applicants: Tsunetoshi Honda, Akita-shi (JP); Daisuke Takano, Akita-shi (JP)

(72) Inventors: Tsunetoshi Honda, Akita-shi (JP); Daisuke Takano, Akita-shi (JP)

(73) Assignees: MITSUBISHI MATERIALS ELECTRONIC CHEMICALS CO., LTD, Akita-shi (JP); MITSUBISHI MATERIALS CORPORATION, Tokyo (JP)

(21) Appl. No.: 13/814,801

(22) PCT Filed: Sep. 21, 2010
(86) PCT No.: PCT/JP2010/066338

§ 371 Date: Feb. 7, 2013

(30) Foreign Application Priority Data
Aug. 11, 2010 (JP) 2010-180251

(51) Int. Cl. C07C 303/38

(52) U.S. Cl. CPC C07C 303/38
USPC 564/96

(57) ABSTRACT

In this process for producing a fluorine-containing sulfonylimide compound, a process for producing a fluorine-containing sulfonylimide compound ((Rf¹SO₂)(Rf²SO₂)₂N.M) is employed that comprises a first step of obtaining a reaction liquid by reacting a perfluoroalkylsulfonyl fluoride (Rf¹SO₂F) with ammonia, a second step of obtaining a mixture containing an alkaline metal salt of a perfluoroalkylsulfonamide (Rf'SO₂NH.M) by reacting the reaction liquid with at least one type of alkaline metal compound selected from among hydroxides, carbonates and bicarbonates of alkaline metals M of either Li, Na or K, and a third step of reacting the mixture with a perfluoroalkylsulfonyl halide (Rf²SO₂X), wherein Rf¹ and Rf² represent linear or branched perfluoroalkyl groups having 1 to 4 carbon atoms, and X represents fluorine or chlorine.

### TECHNICAL FIELD

The present invention relates to an improved process for producing a fluorine-containing sulfonylimide compound.

The present invention claims priority on the basis of Japanese Patent Application No. 2010-180251, filed in Japan on Aug. 11, 2010, the contents of which are incorporated herein by reference.

### BACKGROUND ART

Fluorine-containing sulfonylimide compounds are known to be substances that are useful as anion sources of ion-conducting materials and ionic liquids. In addition, ionic liquids in particular are expected to be used as electrolytes of batteries and capacitors as well as reaction solvents and catalysts, and are commonly known to be obtained by an exchange of salts between a salt of a fluorine-containing sulfonylimide compound in the form of a fluorine-containing sulfonylimide acid and a halide salt of a quaternary amine in the manner of imidazolium bromide salt.

In general, known examples of processes for producing fluorine-containing sulfonylimide compounds include the processes described in Patent Document 1 and Patent Document 2. More specifically, Patent Document 1 discloses a process for producing a perfluoroalkylsulfonylimide salt ((Rf^{a}SO₂)(Rf^{b}SO₂)N.M) by reacting a perfluoroalkylsulfonamide (Rf^{a}SO₂NH₂), a perfluoroalkylsulfonyl halide (Rf^{a}SO₂X), and a fluorine compound (MF) such as potassium fluoride in the presence of an organic solvent such as acetonitrile as shown in the following formula (1).

[Chemical Formula 1]

Rf.SO₂NH₂+Rf^{b}SO₂X+2MF→(Rf.SO₂)(Rf^{b}SO₂)(Rf^{b}SO₂)N.M+MF.HF+HX (1)

In formula (1) above, Re and Rf^{b} represent perfluoroalkyl groups and the like, M represents an alkaline metal and the like, and X represents fluorine or chlorine.

In addition, Patent Document 2 discloses a process for producing a perfluoroalkylsulfonylimide salt ((Rf^{c}SO₂)(Rf^{d}SO₂)N.M) by reacting a perfluoroalkylsulfonamide and a perfluoroalkylsulfonyl fluoride in the presence of a tertiary amine or heterocyclic amine as shown in the following formula (2).

[Chemical Formula 2]

Rf^{c}SO₂F+Rf^{d}SO₂NH₂+2NR¹R²R³→(Rf^{c}SO₂)(Rf^{d}SO₂)N.NHR¹R²R³+R¹R²R³NHF (2)

In formula (2) above, Rf^{c} and Rf^{d} represent perfluoroalkyl groups and the like, and R¹ to R³ represent alkyl groups and the like.

However, in the aforementioned Patent Document 1 and Patent Document 2, a perfluoroalkylsulfonamide is used as a raw material. A known example of a process for producing this perfluoroalkylsulfonamide is described in Non-Patent Document 1, and consists of reacting a perfluoroallcylsulfonyl halide represented by the general formula CₙF₂ₙ₊₁SO₂X (wherein, n represents an integer of 1 to 4, and X represents F or Cl) with ammonia (NH₃) as shown in the following reaction formula (3).

[Chemical Formula 3]

CₙF₂ₙ₊₁SO₂X+2NH₃+CₙF₂ₙ₊₁SO₂NH₂+NH₄.X (3)

More specifically, Non-Patent Document 1 discloses a process for producing trifluoromethanesulfonamide (CF₃SO₂NH₂). The process for producing trifluoromethanesulfonamide disclosed in Non-Patent Document 1 consists of reacting anhydrous ammonia and trifluoromethanesulfonyl fluoride (CF₃SO₂F) while cooling at −78° C. in a solvent-free system, followed by extracting the resulting trifluoromethanesulfonamide with dioxane.

### PRIOR ART DOCUMENTS

### Patent Documents

[Patent Document 1] Japanese Unexamined Patent Application, First Publication No. 2001-288193

[Patent Document 2] Japanese Unexamined Patent Application, First Publication No. H08-81436

### Non-Patent Documents

[Non-Patent Document 1] Inorganic Chemistry, 1984, 23, 3720-3723

### DISCLOSURE OF INVENTION

### Problems to be Solved by the Invention

However, in the processes described in Patent Document 1 and Patent Document 2, when forming a perfluoroalkylsulfonylimide salt by reacting a perfluoroalkylsulfonamide and a perfluoroalkylsulfonyl halide, there was the problem of being required to add a large amount of an expensive alkaline metal fluoride or tertiary amine as an additive of the imidization reaction.

In addition, in the processes described in Patent Document 1 and Patent Document 2, since ammonia gas, anhydrous ammonia (b.p. −33° C.) or an ammonium salt and the like is used for the ammonia that reacts with the perfluoroalkylsulfonyl halide in the production process of the raw material in the form of a perfluoroalkylsulfonamide, there were also problems such as the risks of toxicity and flammability of ammonia, the need for refrigeration equipment and pressurization equipment, and increased production costs.

With the foregoing in view, an object of the present invention is to provide a process for producing a fluorine- containing sulfonylimide compound that is highly safe, highly productive and is simple.

### Means for Solving the Problems

As a result of conducting extensive studies in order to solve the aforementioned problems, the inventors of the present invention found that, by reacting a solution, obtained by reacting ammonia, and preferably aqueous ammonia, with a perfluoroalkylsulfonyl fluoride and dissolving an ammonium salt of the resulting perfluoroalkylsulfonamide with ammonium fluoride, at least one type of compound selected from a hydroxide, carbonate and bicarbonate of an alkaline metal element, and a perfluoroalkylsulfonyl halide, a fluorine-containing sulfonylimide compound can be formed without using an expensive alkaline metal fluoride or tertiary amine, thereby leading to completion of the present invention.

Namely, the present invention employs the constitutions indicated below.

[1] A process for producing a fluorine-containing sulfonylimide compound represented by the following formula (4), comprising:

a first step of obtaining a reaction liquid by reacting a perfluoroalkylsulfonyl fluoride represented by the following formula (5) with ammonia,

a second step of obtaining a mixture containing an alkaline metal salt of a perfluoroalkylsulfonamide represented by the following formula (6) by reacting the reaction liquid with at least one type of alkaline metal compound selected from among hydroxides, carbonates and bicarbonates of alkaline metals M of either Li, Na or K, and

a third step of reacting the mixture with a perfluoroalkylsulfonyl halide represented by the following formula (7):

(Rf¹SO₂)(Rf²SO₂)N.M (4)

Rf¹SO₂F (5)

Rf¹SO₂NH.M (6)

Rf²SO₂X (7)

(wherein, in the formulas (4) to (7), Re and Re represent linear or branched perfluoroalkyl groups having 1 to 4 carbon atoms, and in formula (7), X represents fluorine (F) or chlorine (Cl)).

[2] The process for producing a fluorine-containing sulfonylimide compound represented by formula (4) described in [1] above, comprising:

a first step of obtaining a reaction liquid containing an ammonium salt of a perfluoroalkylsulfonamide represented by the following formula (8) and ammonium fluoride by reacting a perfluoroalkylsulfonyl fluoride represented by formula (5) with ammonia,

a second step of obtaining a mixture containing an alkaline metal salt of a perfluoroalkylsulfonamide represented by formula (6) and an alkaline metal fluoride represented by the following formula (9) by reacting the reaction liquid with at least one type of alkaline metal compound selected from among hydroxides, carbonates and bicarbonates of alkaline metal elements M of either Li, Na or K, and

a third step of reacting the mixture with a perfluoroalkylsulfonyl halide represented by formula (7):

Rf¹SO₂NH.NH₄ (8)

MF (9)

(wherein, in formula (8), Rf1 represents a linear or branched perfluoroalkyl group having 1 to 4 carbon atoms).

[3] The process for producing a fluorine-containing sulfonylimide compound described in [1] or [2] above, wherein,

in the first step, the ammonia is aqueous ammonia and the resulting reaction liquid is an aqueous solution, and

in the second step, the reaction between the reaction liquid and the alkaline metal compound is carried out in the aqueous solution.

[4] The process for producing a fluorine-containing sulfonylimide compound described in any one of [1] to [3] above, further comprising a fourth step of recovering ammonia generated in the second step and supplying to the first step.

The process for producing a fluorine-containing sulfonylimide compound described in any one of [1] to [4] above, wherein the concentration of the aqueous ammonia is within the range of 1% to 50%.

[6] The process for producing a fluorine-containing sulfonylimide compound described in any one of [1] to [5] above, wherein the molar amount of the aqueous ammonia is within the range of 3 to 20 times that of the perfluoroalkylsulfonyl fluoride.

[7] The process for producing a fluorine-containing sulfonylimide compound described in any one of [1] to [6] above, wherein the temperature of the reaction between the perfluoroalkylsulfonyl fluoride and the aqueous ammonia is within the range of 0° C. to 70° C.,

### Effects of the Invention

According to the process for producing a fluorine-containing sulfonylimide compound of the present invention, a constitution is employed in which a prescribed alkaline metal compound is reacted in a reaction liquid obtained by reacting a perfluoroalkylsulfonyl fluoride with ammonia to form a mixture containing an alkaline metal salt of a perfluoroalkylsulfonamide, and this mixture is reacted with a perfluoroalkylsulfonyl halide to produce a fluorine-containing sulfonylimide compound. Consequently, the addition of expensive additives such as an alkaline metal fluoride or tertiary amine is not required. Thus, a fluorine-containing sulfonylimide compound can be produced by a process that is highly safe, highly productive and simple.

### BEST MODE FOR CARRYING OUT THE INVENTION

The following provides a detailed description of a process for producing a fluorine-containing sulfonylimide compound in the form of a perfluoroalkylsulfonimide salt as an embodiment that applies the present invention.

The process for producing a perfluoroalkylsulfonimide salt of the present embodiment is a process for producing a perfluoroalkylsulfonimide salt represented by the following formula (10), and the process comprises a first step of obtaining a reaction liquid by reacting a perfluoroalkylsulfonyl fluoride represented by the following formula (11) with ammonia, a second step of obtaining a mixture containing an alkaline metal salt of a perfluoroalkylsulfonamide represented by the following formula (12) by reacting the reaction liquid with at least one type of alkaline metal compound selected from among hydroxides, carbonates and bicarbonates of alkaline metal elements M of either Li, Na or K, and a third step of reacting the mixture with a perfluoroalkylsulfonyl halide represented by the following formula (13):

(Rf¹SO₂)(Rf²SO₂)N.M (10)

Rf¹SO₂F (11)

Rf¹SO₂NH.M (12)

Rf²SO₂X (13)

(wherein, in the formulas (10) to (13), Rf¹ and Rf² represent linear or branched perfluoroalkyl groups having 1 to 4 carbon atoms, and in formula (13), X represents fluorine (F) or chlorine (Cl)),

### Perfluoroalkylsulfonimide Salt

The process for producing a fluorine-containing sulfonylimide compound in the form of a perfluoroalkylsulfonimide salt of the present embodiment is effective for synthesizing symmetrical imide compounds in which Rf¹ and Rf² are the same, and particularly asymmetrical imide compounds in which Rf¹ and Rf² are different.

Examples of the perfluoroalkylsulfonimide salt represented by the aforementioned formula (10) in the case Rf¹ and Rf² are the same (symmetrical structure) include perfluoroalkylsulfonimide salts such as bis(trifluoromethanesulfonyl)imide salts ((CF₃SO₂)₂N.M), bis(pentafluoroethanesulfonyl)imide salts ((C₂F₅SO₂)₂N.M), bis(heptafluoropropanesulfonyl)imide salts ((C₃F₇SO₂)₂N.M), or bis(nonafluorobutanesulfonyl)imide salts ((C₄F₉SO₂)₂N.M). Furthermore, Rf¹ and Rf² of the present embodiment include branched structural isomers in addition to linear structural isomers in the case of having 3 or 4 carbon atoms.

In addition, in the case Rf¹ and Rf² are different (asymmetrical structure), example include pentafluoro-N-[(trifluoromethane)sulfonyl] ethanesulfonylamide salt ((CF₃SO₂)(C₂F₅SO₂)N.M), heptafluoro-N-[(trifluoromethane)sulfonyl]propane sulfonylamide salt ((CF₃SO₂)(C₃F₇SO₂)N.M), nonafluoro-N-[(trifluoromethane)sulfonyl]butanesulfonylamide salt ((CF₃SO₂)(C₄F₉SO₂)N.M), heptafluoro-N-[(pentafluoroethane) sulfonyl] propanesulfonylamide salt ((C₂F₅SO₂)(C₃F₇SO₂)N.M, nonafluoro-N-[(pentafluoroethane)sulfonyl]butane sulfonylamide salt ((C₂F₅SO₂)(C₄F₉SO₂)N.M), and nonafluoro-N-[(heptalluoropropane)sulfonyl]butanesulfonylamide salt ((CF₃F₇SO₂)(C₄F₉SO₂)N.M).

In addition, in the perfluoroalkylsulfonimide salt represented by the aforementioned formula (10) of the present embodiment, the alkaline metal element M is one type of element of either lithium (Li), sodium (Na) or potassium (K). Thus, examples of the perfluoroalkylsulfonimide salt represented by the aforementioned formula (10) obtained according to the production process of the present embodiment include:

bis(trifluoromethanesulfonyl)imide lithium salt, bis(trifluoromethanesulfonyl)imide sodium salt, bis(trifluoromethanesulfonyl)imide potassium salt, bis(pentafluoroethanesulfonyl)imide lithium salt, bis(pentafluoroethanesulfonyl)imide sodium salt, bis(pentafluoroethanesulfonyl)imide potassium salt, bis(heptafluoropropanesulfonyl)imide lithium salt, bis(heptafluoropropanesulfonyl)imide sodium salt, bis(heptafluoropropanesulfonyl)imide potassium salt, bis(nonafluorobutanesulfonyl)imide lithium salt, bis(nonafluorobutanesulfonyl)imide sodium salt, bis(nonafluorobutanesulfonyl)imide potassium salt, pentafluoro-N-[(trifluoromethane)sulfonyl]ethanesulfonyl amide lithium salt, pentafluoro-N-[(trifluoromethane) sulfonyl]ethanesulfonylamide sodium salt, pentafluoro-N-[(trifluoromethane)sulfonyl]ethanesulfonylamide potassium salt, heptafluoro-N-[(trifluoromethane)sulfonyl]propane sulfonylamide lithium salt, heptafluoro-N-[(trifluoromethane)sulfonyl]propanesulfonylamide sodium salt, heptafluoro-N-[(trifluoromethane)sulfonyl]propanesulfonyl amide potassium salt, nonafluoro-N-[(trifluoromethane) sulfonyl]butanesulfonylamide lithium salt, nonafluoro-N-[(trifluoromethane)sulfonyl]butanesulfonylamide sodium salt, nonafluoro-N-[(trifluoromethane)sulfonyl]butanesulfonyl amide potassium salt, heptafluoro-N-[(pentafluoroethane) sulfonyl]propanesulfonylamide lithium salt, heptafluoro-N-[(pentafluoroethane)sulfonyl]propanesulfonylamide sodium salt, heptafluoro-N-[(pentafluoroethane)sulfonyl]propane sulfonylamide potassium salt, nonafluoro-N-[(pentafluoroethane)sulfonyl]butanesulfonylamide lithium salt, nonafluoro-N-[(pentafluoroethane)sulfonyl]butanesulfonylamide sodium salt, nonafluoro-N-[(pentafluoroethane)sulfonyl]butanesulfonylamide potassium salt, nonafluoro-N-[(heptafluoropropane)sulfonyl]butane sulfonylamide lithium salt, nonafluoro-N-[(heptafluoropropane)sulfonyl]butanesulfonylamide sodium salt, and nonafluoro-N-[(heptafluoropropane)sulfonyl]butane sulfonylamide potassium salt.

### First Step

The first step of the present embodiment is a step of obtaining a reaction liquid containing an ammonium salt of a perfluoroalkylsulfonamide, used as the raw material of a perfluoroalkylsulfonimide salt, by reacting a perfluoroalkylsulfonyl fluoride represented by the aforementioned formula (11) with ammonia. More specifically, the perfluoroalkylsulfonyl fluoride represented by formula (11) is reacted with ammonia to obtain a reaction liquid containing an ammonium salt of the perfluoroalkylsulfonamide represented by the following formula (14) and ammonium fluoride (NH₄F).

Rf¹SO₂NH.NH₄ (14)

Ammonia gas or aqueous ammonia can be used for the raw material ammonia.

In the present embodiment, aqueous ammonia is used particularly preferably.

In the case of using aqueous ammonia in the first step, a reaction liquid is obtained by reacting the aforementioned perfluoroalkylsulfonyl fluoride with aqueous ammonia.

Namely, as shown in the following formula (15), the perfluoroalkylsulfonyl fluoride is reacted with aqueous ammonia to form an ammonium salt of the perfluoroalkylsulfonamide represented by the aforementioned formula (14) and ammonium fluoride.

[Chemical Formula 4]

Rf¹SO₂F+3NH₃.H₂O→Rf¹SO₂NH.NH₄+NH₄F+3H₂O (15)

Here, in formulas (14) and (15), Rf¹ represents a linear or branched perfluoroalkyl group having 1 to 4 carbon atoms.

Namely, examples of the perfluoroalkylsulfonyl fluoride represented by the aforementioned formula (11) include trifluoromethanesulfonyl fluoride, pentafluoroethanesulfonyl fluoride, heptafluoropropanesulfonyl fluoride and nonafluorobutanesulfonyl fluoride.

In addition, examples of an ammonium salt of the perfluoroalkylsulfonamide represented by the aforementioned formula (14) include trifluoromethanesulfonamide ammonium salt, pentafluoroethanesulfonamide ammonium salt, heptafluoropropanesulfonamide ammonium salt and nonafluorobutanesulfonamide ammonium salt.

The lower limit of the concentration of the aqueous ammonia is preferably 1% or more, more preferably 5% or more and even more preferably 10% or more. In addition, the upper limit of the concentration of the aqueous ammonia is preferably 50% or less, more preferably 40% or less and even more preferably 30% or less. If the concentration of the aqueous ammonia is less than 1%, there is a shortage of ammonia to react with the perfluoroalkylsulfonyl fluoride and the perfluoroalkylsulfonyl fluoride ends up undergoing hydrolysis, thereby making this undesirable. On the other hand, if the concentration of the aqueous ammonia exceeds 50%, it becomes difficult to form aqueous ammonia, thereby making this undesirable.

In contrast, if the concentration of the aqueous ammonia is within the aforementioned ranges, it is easy to prepare aqueous ammonia and there is little hydrolysis of the perfluoroalkylsulfonyl fluoride, thereby making this desirable.

In addition, the molar amount of the aqueous ammonia is preferably within the range of 3 to 20 times, and more preferably within the range of 5 to 10 times, that of the perfluoroalkylsulfonyl fluoride.

If the molar amount of aqueous ammonia is less than 3 times that of the perfluoroalkylsulfonyl fluoride, the reaction of the aforementioned formula (15) becomes inadequate, thereby making this undesirable. On the other hand, if the molar amount of aqueous ammonia exceeds 20 times that of the perfluoroalkylsulfonyl fluoride, the reaction becomes economically wasteful. In contrast, if the molar amount of the aqueous ammonia is within the aforementioned ranges, the reaction of formula (15) proceeds adequately and hydrolysis of the perfluoroalkylsulfonyl fluoride is inhibited, thereby making this desirable.

In addition, in the reaction step, the temperature of the reaction between the perfluoroalkylsulfonyl fluoride and the aqueous ammonia is preferably controlled to be within the range of 0° C. to 70° C. If the temperature of the reaction between the perfluoroalkylsulfonyl fluoride and the aqueous ammonia is lower than 0° C., the amidation reaction proceeds slowly and the ratio of unreacted raw material in the form of sulfonyl fluoride ends up being lost as gas, thereby making this undesirable. On the other hand, if the reaction temperature exceeds 70° C., solubility in aqueous ammonia decreases, and the ratio of ammonia lost as gas increases, thereby making this undesirable.

### Second Step

The second step of the present embodiment is a step of obtaining a mixture containing an alkaline metal salt of a perfluoroalkylsulfonamide represented by the aforementioned formula (12) and an alkaline metal fluoride represented by the following formula (16) by reacting the aforementioned reaction liquid obtained in the first step with at least one type of alkaline metal compound selected from among hydroxides, carbonates and bicarbonates of alkaline metal elements M of either Li, Na or K.

MF (16)

In the case the reaction liquid obtained in the aforementioned first step is an aqueous ammonia solution in the second step, the reaction between this reaction liquid and the aforementioned alkaline metal compound is carried out in an aqueous ammonia solution.

Here, examples of at least one type of alkaline metal compound selected from among hydroxides (MOH), carbonates (M₂CO₃) and bicarbonates (MHCO₃) of alkaline metal elements M of either Li, Na or K in the present embodiment include lithium hydroxide (LiOH), sodium hydroxide (NaOH), potassium hydroxide (KOH), lithium carbonate (Li₂CO₃), sodium carbonate (Na₂CO₃), potassium carbonate (K₂CO₃), lithium hydrogen carbonate (LiHCO₃), sodium hydrogen carbonate (NaHCO₃) and potassium hydrogen carbonate (KHCO₃).

Namely, in the second step, as shown in the following formulas (17) to (19), an ammonium salt of a perfluoroalkylsulfonamide and ammonium fluoride (NH₄F) dissolved in an aqueous solution are reacted with the aforementioned alkaline metal compound to form a mixture containing an alkaline metal salt of the perfluoroalkylsulfonamide represented by the aforementioned formula (12) and the alkaline metal fluoride represented by the aforementioned formula (16).

The following formula (17) is a reaction formula in the case of using a hydroxide (MOH) of an alkaline metal element M for the aforementioned alkaline metal compound.

[Chemical Formula 5]

Rf¹SO₂NH.NH₄+NH₄F+2MOH→Rf¹SO₂NH.M+MF+2NH₃↑+2H₂O (17)

In addition, the following formula (18) is a reaction formula in the case of using a carbonate (M₂CO₃) of an alkaline metal element M for the aforementioned alkaline metal compound.

[Chemical Formula 6]

Rf¹SO₂NH.NH₄+NH₄F+M₂CO₃→Rf¹SO₂NH.M+MF+2NH₃↑+CO₂↑+H₂O (18)

Moreover, the following formula (19) is a reaction formula in the case of using a bicarbonate (MHCO₃) of an alkaline metal element M for the aforementioned alkaline metal compound.

[Chemical Formula 7]

Rf¹SO₂NH.NH₄+NH₄F+2MHCO₃→Rf¹SO₂NH.M+MF+2NH₃↑+2CO₂↑+2H₂O (19)

In formula (12) above, Rf¹ represents a linear or branched perfluoroalkyl group having 1 to 4 carbon atoms.

Namely, examples of alkaline metal salts of the perfluoroalkylsulfonamide represented by the aforementioned formula (12) include trifluoromethanesulfonamide lithium salt, trifluoromethanesulfonamide sodium salt, trifluoromethanesulfonamide potassium salt, pentafluoroethanesulfonamide lithium salt, pentafluoroethanesulfonamide sodium salt, pentafluoroethanesulfonamide potassium salt, heptafluoropropanesulfonamide lithium salt, heptafluoropropanesulfonamide sodium salt, heptafluoropropanesulfonamide potassium salt, nonafluorobutanesulfonamide lithium salt, nonafluorobutanesulfonamide sodium salt and nonafluorobutanesulfonamide potassium salt.

In addition, examples of the alkaline metal fluoride represented by the aforementioned formula (16) include lithium fluoride (LiF), sodium fluoride (NaF) and potassium fluoride (KF).

An alkaline metal salt of the perfluoroalkylsulfonamide serving as raw material of the perfluoroalkylsulfonimide salt is formed in the manner described above.

### Third Step

The third step of the present embodiment is a step of reacting the mixture obtained in the second step with a perfluoroalkylsulfonyl halide represented by the aforementioned formula (13). More specifically, an aqueous solution containing the mixture obtained in the second step is concentrated to obtain a mixture containing an alkaline metal salt of the perfluoroalkylsulfonamide and an alkaline metal fluoride. This mixture is then reacted with the aforementioned perfluoroalkylsulfonyl halide in a solvent to form the perfluoroalkylsulfonimide salt represented by the aforementioned formula (10).

Here, there are no particular limitations on solvents able to be used in the third step provide they are inert with respect to the alkaline metal salt of the perfluoroalkylsulfonamide and the alkaline metal fluoride. Examples of such solvents include ethers such as diethyl ether, tetrahydrofuran (THF), 1,4-dioxane, diisopropyl ether, ethylene glycol dimethyl ether, diethylene glycol dimethyl ether, triethylene glycol dimethyl ether or tetraethylene glycol dimethyl ether, halogenated hydrocarbons such as dichloromethane, dichloroethane or perfluorocarbon, hydrocarbons such as benzene, heptane or hexane, carbonate-based solvents such as dimethyl carbonate or diethyl carbonate, nitrile-based solvents such as acetonitrile, propionitrile, butyronitrile, malononitrile or adiponitrile, amides such as N,N-dimethylformamide or N,N-dimethylacetoamide, dimethylsulfoxide and sulfolan. These solvents can be used alone or as a mixture.

In the aforementioned formula (13), Re represents a linear or branched perfluoroalkyl group in the same manner as the previously described Re. In addition, X represents fluorine (F) or chlorine (Cl).

Namely, examples of the perfluoroalkylsulfonyl halide represented by the aforementioned formula (13) include the perfluoroalkylsulfonyl fluoride represented by the aforementioned formula (11), trifluoromethanesulfonyl chloride, pentafluoroethanesulfonyl chloride, heptafluoropropanesulfonyl chloride and nonafluorobutanesulfonyl chloride,

In the third step, the aforementioned perfluoroalkylsulfonyl halide is reacted with an alkaline meal salt of a perfluoroalkylsulfonamide and an alkaline metal fluoride in a solvent.

Namely, as shown in the following formula (20), an alkaline metal salt of a perfluoroalkylsulfonamide is reacted with a perfluoroalkylsulfonyl halide to form the perfluoroalkylsulfonimide salt represented by the aforementioned formula (10).

[Chemical Formula 8]

Rf¹SO₂NH.M+MF+Rf²SO₂F→(Rf¹SO₂)(Rf²SO₂)N.M+MF.HF (20)

In the production process of the present embodiment, a combination of the perfluoroalkylsulfonyl fluoride represented by formula (11) and the perfluoroalkylsulfonyl halide represented by formula (13) can be suitably selected in order to produce the desired perfluoroalkylsulfonimide salt represented by the aforementioned formula (10).

Namely, a perfluoroalkylsulfonimide salt having a symmetrical structure can be obtained by using perfluoroalkyl groups having the same structure (Rf¹=Rf²) in the perfluoroalkylsulfonyl fluoride represented by formula (11) and the perfluoroalkylsulfonyl halide represented by formula (13).

On the other hand, a perfluoroalkylsulfonimide salt having an asymmetrical structure can be obtained by using perfluoroalkyl groups having different structures in the perfluoroalkylsulfonyl fluoride represented by formula (11) and the perfluoroalkylsulfonyl halide represented by formula (13).

### Fourth Step

Moreover, a fourth step is preferably provided in the production process of the present embodiment for recovering ammonia gas generated in the second step, forming aqueous ammonia from the ammonia gas, and supplying the aqueous ammonia to the first step. More specifically, ammonia gas generated in the aforementioned formulas (17) to (19) of the second step is captured in water and recovered in the form of aqueous ammonia. Thus, according to the production process of the present embodiment, by providing the fourth step, ammonia remaining in the reaction system or ammonium salt formed as a by-product is not discarded, but rather can be reused as aqueous ammonia used in the first step.

However, in conventional processes for producing a perfluoroalkylsulfonimide salt, the perfluoroalkylsulfonamide serving as raw material of the perfluoroalkylsulfonimide salt was typically formed and used by reacting a perfluoroalkylsulfonyl halide with anhydrous ammonia in the presence of an ether solvent (refer to the aforementioned Non-Patent Document 1). Moreover, in order to isolate the perfluoroalkylsulfonamide formed in the aforementioned process, hydrochloric acid was added following the reaction between the perfluoroalkylsulfonyl halide and the anhydrous ammonia, and the perfluoroalkylsulfonamide was dissolved in the ether layer while the ammonium halide formed as a by-product was dissolved in the hydrochloric acid layer, thereby separating the perfluoroalkylsulfonamide and the ammonium halide. The isolated perfluoroalkylsulfonamide was then purified and used as a raw material for imidization, while the ammonium halide formed as a by-product was discarded after being separated and removed from the reaction system.

Here, when synthesizing a perfluoroalkylsulfonimide salt, the imidization reaction does not proceed efficiently with only a perfluoroalkylsulfonamide and perfluoroalkylsulfonyl halide, and in the conventional production processes of a perfluoroalkylsulfonimide salt indicated in the aforementioned Patent Documents 1 and 2, a constitution is employed in which a base such as an alkaline metal salt or tertiary amine is added to raw materials consisting of a perfluoroalkylsulfonamide and perfluoroalkylsulfonyl halide and allowed to react.

However, in the conventional production processes of a perfluoroalkylsulfonimide salt as indicated in the aforementioned Patent Documents 1 and 2, it was necessary to add a large amount of additional raw materials in the form of an alkaline metal fluoride and tertiary amine serving as additives of the imidization reaction. Since these additives are expensive, there was the problem of considerable production costs.

In contrast, according to the process for producing a perfluoroalkylsulfonimide salt of the present embodiment, as a result of reacting a prescribed alkaline metal compound while the reaction product of a perfluoroalkylsulfonyl fluoride and ammonia remains mixed in solution, a mixture of the respectively corresponding alkaline metal salt of a perfluoroalkylsulfonamide and alkaline metal fluoride can be formed from an ammonium salt of the perfluoroalkylsulfonamide and ammonium fluoride. In this manner, since a perfluoroalkylsulfonamide salt can be formed by using an inexpensive alkaline metal compound, it is not necessary to use expensive additives such as an alkaline metal fluoride or tertiary amine.

A perfluoroalkylsulfonimide salt can then be formed by reacting the resulting mixture, namely the alkaline metal salt of the perfluoroalkylsulfonamide and the alkaline metal fluoride.

In this manner, by reacting with an inexpensive alkaline metal compound without removing ammonium fluoride formed as a by-product when forming an ammonium salt of the perfluoroalkylsulfonamide from the reaction system, alkaline metal fluoride can be formed within the reaction system. Consequently, expensive additives in the manner of alkaline metal fluoride or tertiary amine are not required to be added separately as a portion of the raw materials.

Furthermore, an alkaline metal fluoride is present during the imidization reaction in both the production process of the present embodiment and the production process described in Patent Document 1. However, as has been previously described, in contrast to the alkaline metal fluoride in Patent Document I being newly added from outside the reaction system as a portion of the raw materials, the alkaline metal fluoride in the production process of the present embodiment is formed from within the reaction system instead of being newly added from outside the reaction system. Namely, the alkaline metal fluoride in the production process of the present embodiment is completely different from the alkaline metal fluoride in Patent Document 1.

As has been explained above, according to the production process of the present embodiment, a perfluoroalkylsulfonimide salt can be produced by a method that is highly safe, highly productive and simple.

According to the process for producing a fluorine-containing sulfonylimide compound of the present invention, the first step employs a constitution in which a perfluoroalkylsulfonyl fluoride is reacted with ammonia, and preferably aqueous ammonia, to obtain a reaction liquid containing an ammonium salt of a perfluoroalkylsulfonamide and ammonium fluoride. In this manner, when obtaining the aforementioned reaction liquid, since the use of aqueous ammonia eliminates the need to use ammonia gas or anhydrous ammonia, a reaction liquid containing an ammonium salt of a perfluoroalkylsulfonamide and ammonium fluoride can be obtained by a process that is highly safe, highly productive and simple.

In addition, the second step employs a constitution in which the aforementioned reaction liquid and a prescribed alkaline metal compound are reacted to obtain a mixture containing an alkaline metal salt of a perfluoroalkylsulfonamide and an alkaline metal fluoride. In this manner, in addition to forming an alkaline metal salt of a perfluoroalkylsulfonamide from the ammonium salt of a perfluoroalkylsulfonamide formed in the first step, since an alkaline metal fluoride can he formed from ammonium fluoride and an alkaline metal compound, the ammonium fluoride formed as a by-product when forming the ammonium salt of the perfluoroalkylsulfonamide can be effectively used without having to separate and remove.

Moreover, the third step employs a constitution in which the aforementioned mixture is reacted with a perfluoroalkylsulfonyl halide. Here, since the alkaline metal fluoride formed in the second step is present in this mixture, it is not necessary to newly add expensive additives in the manner of an alkaline metal fluoride or tertiary amine required during the imidization reaction.

Moreover, in the case of carrying out the reaction between the aforementioned reaction liquid and the aforementioned alkaline metal compound in an aqueous solution in the second step, a highly safe process for producing a fluorine-containing sulfonylimide compound can be provided without using an organic solvent when forming the ammonium salt of the perfluoroalkylsulfonamide.

In addition, according to the process for producing a fluorine-containing sulfonylimide compound of the present invention, since a constitution is employed that provides a fourth step of recovering ammonia gas generated in the aforementioned second step, forming aqueous ammonia from this ammonia gas and supplying the aqueous ammonia to the first step, ammonia produced as a by-product when forming the mixture containing an alkaline metal salt of a perfluoroalkylsulfonamide and an alkaline metal fluoride can be reused to realize a process for producing a fluorine-containing sulfonylimide compound that is industrially advantageous.

Furthermore, the technical scope of the present invention is not limited to the aforementioned embodiment, but rather can be modified in various ways within a range that does not deviate from the gist of the present invention. For example, although an explanation was provided of the case of using aqueous ammonia for the ammonia in the first step of the aforementioned embodiment, ammonia gas can also be used.

In the case of using ammonia gas in the first step, the perfluoroalkylsulfonyl fluoride and the ammonia gas are reacted in an organic solvent such as ether, and an ammonium salt of the perfluoroalkylsulfonamide can be obtained along with ammonium fluoride that has precipitated in the form of a slurry.

In the second step, a prescribed alkaline metal compound may be added to an organic solvent following the reaction of the first step, or water may be added and reacted after forming an organic solvent-water system. In addition, water may be added after initially concentrating the organic solvent, and then reacted with the alkaline metal compound in an aqueous solution system. Regardless of which of these methods is used, a mixture can be obtained that contains an alkaline metal salt of a perfluoroalkylsulfonamide and an alkaline metal fluoride in the same manner as the aforementioned embodiment.

### EXAMPLES

The following provides a more detailed explanation of the present invention through examples thereof. Furthermore, the present invention is not limited in any way by these examples.

### Example 1

### Synthesis of (C₃F₇SO₂)₂NK

First, 50 g of 20% aqueous ammonia were placed in a 100 ml flask, followed by dropping in 30 g of heptafluoropropanesulfonyl fluoride (C₃F₇SO₂F) at 40° C. After stirring for 2 hours at 40° C., 28 g of a 48% aqueous solution of potassium hydroxide (KOH) were added and concentrated.

Next, 8.1 g of the concentrated reaction mixture of heptafluoropropanesulfonamide and potassium hydroxide and 5.7 g of heptafluoropropanesulfonyl fluoride (C₃F₇SO₂F) were placed in a 100 ml flask, followed by stirring for 48 hours at 40° C. using 32 g of acetonitrile as solvent, 8.6 g of bis(heptafluoropropanesulfonyl)imide potassium salt (C₃F₇SO₂)₂NK were obtained as determined by NMR using an internal standard. Yield based on heptafluoropropanesulfonyl fluoride (C₃F₇SO₂F) was 86%,

### Example 2

### Synthesis of (C₃F₇SO₂)(C₄F₉SO₂)NK

5.0 g of a reaction mixture of heptafluoropropanesulfonamide and potassium hydroxide obtained in the same manner as Example 1 and 4.1 g of nonafluorobutanesulfonyl fluoride (C₄F₉SO₂F) were combined, followed by stirring for 30 hours at 50° C. using 20 g of acetonitrile as solvent.

5.45 g of nonafluoro-N-[(heptafluoropropane)sulfonyl] butanesulfonylamide potassium salt ((C₃F₇SO₂)(C₄F₉SO₂)NK) were obtained as determined by NMR using an internal standard. Yield based on heptafluoropropanesulfonyl fluoride (C₃F₇SO₂F) was 83%.

### Example 3

### Synthesis of (C₂F₅SO₂)(C₃F₇SO₂)NK

5.0 g of a reaction mixture consisting of heptafluoropropanesulfonamide and potassium hydroxide obtained in the same manner as Example 1 and 2.8 g of pentafluoroethanesulfonyl fluoride (C₂F₅SO₂F) were blown in at 0° C., followed by stirring for 48 hours at 50° C. using 20 g of acetonitrile as solvent. 4.39 g of heptafluoro-N-[(pentafluoroethane)sulfonyl]propanesulfonylamide potassium salt ((C₂F₅SO₂)(C₃F₇SO₂)NK) were obtained. Yield based on heptafluoropropanesulfonyl fluoride (C₃F₇SO₂F) was 81%.

### Example 4

### Recovery of Ammonia

First, 150 g of 20% aqueous ammonia were placed in a 200 ml flask, and the aqueous ammonia was circulated through an SUS reaction column packed with a packing, followed by blowing in 36.5 g of trifluoromethanesulfonyl fluoride (CF₃SO₂F) at 25° C. and stirring for 2 hours at 25° C.

Continuing, 40 g of a 48% aqueous solution of potassium hydroxide were dropped in followed by stirring for 1 hour at 100° C., and when 87 g of water were recovered by trapping the generated ammonia gas, 110 g of 20% aqueous ammonia were obtained (anhydrous ammonia: 21.6 g, recovery rate: 90%).

Subsequently, concentration of the reaction liquid consisting of trifluoromethanesulfonamide and potassium hydroxide resulted in the obtaining of 43 g of the reaction mixture of trifluoromethanesulfonamide and potassium hydroxide.

1. A process for producing a fluorine-containing sulfonylimide compound represented by the following formula (1), comprising:
a first step of obtaining a reaction liquid by reacting a perfluoroalkylsulfonyl fluoride represented by the following formula (2) with ammonia,
a second step of obtaining a mixture containing an alkaline metal salt of a perfluoroalkylsulfonamide represented by the following formula (3) by reacting the reaction liquid with at least one type of alkaline metal compound selected from among hydroxides, carbonates and bicarbonates of alkaline metals M of either Li, Na or K, and
a third step of reacting the mixture with a perfluoroalkylsulfonyl halide represented by the following formula (4):
(Rf¹SO₂)(Rf²SO₂)N.M (1)
Rf¹SO₂F (2)
Rf¹SO₂NH.M (3)
Rf²SO₂X (4)
(wherein, in the formulas (1) to (4), Rf¹ and Rf² represent linear or branched perfluoroalkyl groups having 1 to 4 carbon atoms, and in formula (4), X represents fluorine (F) or chlorine (Cl)).

2. The process for producing the fluorine-containing sulfonylimide compound represented by formula (1) according to claim 1, comprising:
a first step of obtaining a reaction liquid containing an ammonium salt of a perfluoroalkylsulfonamide represented by the following formula (5) and ammonium fluoride by reacting the perfluoroalkylsulfonyl fluoride represented by formula (2) with ammonia,
a second step of obtaining a mixture containing an alkaline metal salt of the perfluoroalkylsulfonamide represented by formula (3) and an alkaline metal fluoride represented by the following formula (6) by reacting the reaction liquid with at least one type of alkaline metal compound selected from among hydroxides, carbonates and bicarbonates of alkaline metal elements M of either Li, Na or K, and
a third step of reacting the mixture with the perfluoroalkylsulfonyl halide represented by formula (4):
Rf¹SO₂NH.NH₄ (5)
MF (6)
(wherein, in formula (5), Rf¹ represents a linear or branched perfluoroalkyl group having 1 to 4 carbon atoms).

3. The process for producing a fluorine-containing sulfonylimide compound according to claim 1, wherein,
in the first step, the ammonia is aqueous ammonia and the resulting reaction liquid is an aqueous solution, and
in the second step, the reaction between the reaction liquid and the alkaline metal compound is carried out in the aqueous solution.

4. The process for producing a fluorine-containing sulfonylimide compound according to claim 1, further comprising a fourth step of recovering ammonia generated in the second step and supplying to the first step.

5. The process for producing a fluorine-containing sulfonylimide compound according to claim 1, wherein the concentration of the aqueous ammonia is within the range of 1% to 50%.

6. The process for producing a fluorine-containing sulfonylimide compound according to claim 1, wherein the molar amount of the aqueous ammonia is within the range of 3 to 20 times that of the perfluoroalkylsulfonyl fluoride.

7. The process for producing a fluorine-containing sulfonylimide compound according to claim 1, wherein the temperature of the reaction between the perfluoroalkylsulfonyl fluoride and the aqueous ammonia is within the range of 0° C. to 70° C.
